(19) United States
(12) Patent Application Publication
(10) Pub. No.: US 2026/0104469 A1
(43) Pub. Date: Apr. 16, 2026

(54) SYSTEM TO QUANTIFY DEGRADATION OF A BATTERY CELL TO PREDICT CELL PERFORMANCE METRICS

(71) Applicant: GM Global Technology Operations LLC, Detroit, MI (US)

(72) Inventors: Mingjie Tu, Sterling Heights, MI (US); Thanh-Son Dao, Rochester Hills, MI (US); Jingyuan Liu, Shanghai (CN); Louis G. Hector, JR., Shelby Twp, MI (US); Lei Wang, Rochester Hills, MI (US); Gongshin Qi, Troy, MI (US); Vamakshi Yadav, Sterling Heights, MI (US); Raneen Taha, Sterling Heights, MI (US); Jing Gao, Rochester, MI (US); Brian J. Koch, Berkley, MI (US)

(21) Appl. No.: 18/922,743

(22) Filed: Oct. 22, 2024

(30) Foreign Application Priority Data
Oct. 11, 2024 (CN) 202411418803.9

(51) Int. Cl. G01R 31/392; G01R 31/367; G01R 31/378; G01R 31/3835; G01R 31/389; G01R 31/396

(52) U.S. Cl. CPC G01R 31/392; G01R 31/367; G01R 31/378; G01R 31/3835; G01R 31/389; G01R 31/396

(57) ABSTRACT

A method for determining degradation of a battery cell with a lithium-manganese-rich (LMR) cathode is provided. The method includes measuring open circuit voltage (OCV) of a battery cell during cycling; predicting OCV shifting; determining OCV hysteresis changes of the battery cell; determining cell voltage decay with an accurate state of charge (SOC); measuring carbon dioxide (CO₂) within the battery cell; measuring gas compositions within the battery cell; estimating a loss of cyclable active material (LAM); fitting the set of reaction rate constants to a rate limited kinetic model and a diffusion limited model; determining solid electrolyte interphase (SEI) and metallic Li thicknesses on the anode and cathode electrolyte interphase (CEI) thickness on the cathode; determining cell resistance and voltage drop; and predicting performance metrics using an electrochemical model to obtain a remaining useful battery cell life, battery cell state of health (SOH), cell voltage evolution, and cell resistance and impedance.

### INTRODUCTION

The present disclosure relates to a method of analyzing a battery cell, and more specifically to methods for predicting degradation of an LMR battery cell by measuring cell performance metrics.

To power electric motors in electric vehicles, battery packs comprised of numerous battery cells are utilized. Most battery cells in the battery packs can maintain a charge suitable to power the vehicle over a range of several hundred miles. However, over many charge cycles, the battery cells degrade and may be unable to hold a sufficient charge. One common reason for a low-quality battery cell can be an insufficient Solid Electrolyte Interphase (SEI) deposited on the anode of the battery cell. The SEI is formed by the decomposition of electrolyte solvents, additives, and salts. Other factors may also affect the ability of the battery cells to hold a sufficient electrical charge.

Some current practices to analyze the quality of battery cells include performing a discharge capacity check (i.e., checking that the cell provides capacity (measured in amp-hours) that is within a determined specification) and performing an inventory hold, and Open Circuit Voltage (OCV) monitoring (which involves holding the inventory and checking for a decrease in OCV over time). While effective, such quality control measures may be time intensive (with the potential for large quality spills and the added cost of overhead to store inventory) and data poor (i.e., not diagnostic or prognostic). Other methods of analyzing the quality of battery cells involve analyzing the SEI on the anode. However, the battery cell must be cut open (destroying the battery cell) to analyze the SEI.

Thus, while current battery cell degradation prediction methods achieve their intended purpose, there is a need for a new and improved method for predicting battery cell degradation.

### SUMMARY

According to several aspects of the present disclosure, a method for determining degradation of a battery cell with a lithium-manganese-rich (LMR) cathode is provided. The method includes measuring open circuit voltage (OCV) of a battery cell during cycling to obtain testing data; predicting OCV shifting by integrating the testing data into a multi-site multi-reaction (MSMR) framework to obtain MSMR data; determining OCV hysteresis changes of the LMR battery cell at different phases of voltage activation of the battery cell; determining cell voltage decay with an accurate state of charge (SOC) using the MSMR data and the OCV hysteresis changes; measuring carbon dioxide (CO₂) within the battery cell to determine a set of electrolyte consumption data for the battery cell; measuring gas compositions within the battery cell to determine a set of solid electrolyte interphase (SEI)/cathode electrolyte interphase (CEI) growth and lithium (Li) plating data for the battery cell; estimating a loss of cyclable active material (LAM) by using stoichiometric ratio shifts between a cathode and an anode in the battery cell based on the testing data to obtain a set of reaction rate constants; fitting the set of reaction rate constants to a rate limited kinetic model and a diffusion limited model to obtain a set of kinetic model parameters and to obtain a set of diffusion model parameters; determining solid electrolyte interphase (SEI) and metallic Li thicknesses on the anode and cathode electrolyte interphase (CEI) thickness on the cathode using the kinetic model parameters; determining cell resistance and voltage drop using the solid electrolyte interphase (SEI) and metallic Li thicknesses and porosity decrease; and predicting performance metrics using an electrochemical model to obtain a remaining useful battery cell life, battery cell state of health (SOH), cell voltage evolution, and cell resistance and impedance.

In accordance with another aspect of the disclosure, measuring the open circuit voltage (OCV) of a battery cell during cycling includes performing an OCV scan once every 10 cycles for a first 50 cycles and then performing an OCV scan once every 100 cycles until an end of life of the battery cell to obtain testing data.

In accordance with another aspect of the disclosure, the method measuring the open circuit voltage (OCV) of a battery cell during cycling includes three-electrode testing, LMR half-cell OCVs, and mini-sweep cycling.

In accordance with another aspect of the disclosure, the method predicting OCV shifting by integrating the testing data into a multi-site multi-reaction (MSMR) framework to obtain MSMR data includes smoothing test data and generating dQ/dV curves for peak identification.

In accordance with another aspect of the disclosure, the method predicting OCV shifting includes quantifying OCV shifts based on peak location shifts in dQ/dV curves and determining OCV hysteresis gaps and midpoints.

In accordance with another aspect of the disclosure, the method determining OCV hysteresis changes of the LMR battery cell at different phases of voltage activation of the battery cell includes determining OCV hysteresis changes at 3.8 volts, 4.0 volts, 4.2 volts, 4.4 volts, and at 4.6 volts.

In accordance with another aspect of the disclosure, the method estimating a loss of cyclable active material (LAM) includes correlating CO₂ volume with electrolyte self-decomposition and a decrease in electrolyte volume to identify dry out conditions and loss of utilized electrode area.

In accordance with another aspect of the disclosure, the method estimating a loss of cyclable active material (LAM) includes correlating measured gas compositions with SEI growth, Li plating, and consumed electrolyte using NMR analysis, and wherein the gas compositions include at least one of ethylene (C₂H₄), ethane (C₂H₆), or hydrogen gas (H₂).

In accordance with another aspect of the disclosure, the method fitting the set of reaction rate constants to a rate limited kinetic model and a diffusion limited model to obtain a set of kinetic model parameters and to obtain a set of diffusion model parameters includes minimizing squared errors between cell capacities from simulation and test measurements.

In accordance with another aspect of the disclosure, the method further includes defining a life cycling operation to include charging the battery cell by increasing a cell voltage up to approximately 4.2 volts and discharging the battery cell to reduce the cell voltage from approximately 4.2 volts down to approximately 2.7 volts.

In accordance with another aspect of the disclosure, the cathode is formed from a material having the formula xLi₂MnO₃·(1-x)LiMO₂, where M represents at least one of nickel (Ni), cobalt (Co), or manganese (Mn), and where x is a proportion of a lithium-manganese oxide component.

In accordance with another aspect of the disclosure, the method the anode is formed from at least one of graphite, SiOx, or Si.

According to several aspects of the present disclosure, a method for determining quality of a battery cell having a lithium manganese-rich (LMR) cathode is provided. The method includes measuring open circuit voltage (OCV) of a battery cell to obtain testing data; integrating the testing data into a multi-site multi-reaction (MSMR) framework to obtain MSMR data and to predict OCV shifting; determining OCV hysteresis changes of the LMR battery cell at different phases of voltage activation of the battery cell; determining cell voltage decay with an accurate state of charge (SOC) using the MSMR data and the OCV hysteresis changes; measuring gas compositions every 100 charging cycles of the battery cell to obtain a set of reaction rate constants; determining a set of electrolyte consumption data and a set of SEI/CEI growth and lithium (Li) plating data for the battery cell based on the set of reaction rate constants; determining a loss of cyclable active material (LAM) by obtaining a set of stoichiometric coefficients using stoichiometric ratio shifts between a cathode and an anode in the battery cell; determining cell resistance and voltage drop, remaining useful battery cell life, and battery cell state of health (SOH) based on the SEI/CEI growth and lithium (Li) plating data and based on the MSMR data and the OCV hysteresis changes.

In accordance with another aspect of the disclosure, the method measuring the open circuit voltage (OCV) of a battery cell includes performing an OCV scan once every 10 cycles for a first 50 cycles and then performing an OCV scan once every 100 cycles until an end of life of the battery cell to obtain the testing data.

In accordance with another aspect of the disclosure, the method determining OCV hysteresis changes includes smoothing test data and generating dQ/dV curves for peak identification.

In accordance with another aspect of the disclosure, the method determining OCV hysteresis changes of the LMR battery cell includes determining OCV hysteresis changes at 3.8 volts, 4.0 volts, 4.2 volts, 4.4 volts, and at 4.6 volts.

In accordance with another aspect of the disclosure, the method determining a loss of cyclable active material (LAM) includes correlating CO₂ volume with electrolyte self-decomposition and a decrease in electrolyte volume to identify dry out conditions and loss of utilized electrode area.

In accordance with another aspect of the disclosure, the method includes a cathode formed from a material having the formula xLi₂MnO₃·(1-x)LiMO₂, where M represents at least one of nickel (Ni), cobalt (Co), or manganese (Mn), and where x is a proportion of a lithium-manganese oxide component.

In accordance with another aspect of the disclosure, the method includes an anode formed from at least one of graphite, silicon oxide (SiOx), or silicon (Si).

According to several aspects of the present disclosure, a method for determining quality of a battery cell having a lithium manganese-rich (LMR) cathode is provided. The method includes integrating open circuit voltage (OCV) measurements of a battery cell into a multi-site multi-reaction (MSMR) framework to obtain MSMR data and to predict OCV shifting; determining cell voltage decay with an accurate state of charge (SOC) using the MSMR data and OCV shifting; measuring gas compositions during charging cycles of the battery cell to obtain a set of reaction rate constants; determining a set of electrolyte consumption data and a set of SEI/CEI growth and lithium (Li) plating data for the battery cell based on the set of reaction rate constants; determining a loss of cyclable active material (LAM) by finding a set of stoichiometric coefficients using stoichiometric ratio shifts between a cathode and an anode in the battery cell; and determining cell resistance and voltage drop, remaining useful battery cell life, and battery cell state of health (SOH) based on the SEI/CEI growth and lithium (Li) plating data and based on the MSMR data and OCV hysteresis changes.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a diagram of a system including a battery cell showing cell components of a cathode, an anode, a positive current collector, and a negative current collector and a degradation determination system in communication with the battery cell, in accordance with the present disclosure.

FIG. 2 is a schematic diagram illustrating the degradation determination system shown in FIG. 1, in accordance with the present disclosure.

FIG. 3 is a flow diagram illustrating a method for determining lithium manganese-rich (LMR) quality of a battery cell with a lithium-manganese-rich (LMR) cathode, in accordance with the present disclosure.

### DETAILED DESCRIPTION

The following description is merely exemplary in nature and is not intended to limit the present disclosure, application, or uses.

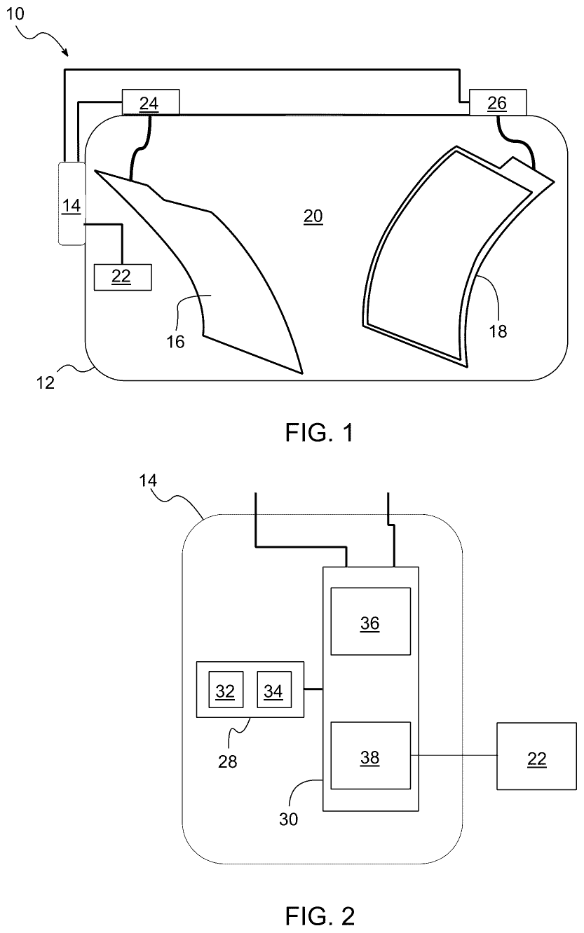

Referring to FIG. 1, a schematic of a system 10 for determining battery cell quality is shown. The system 10 generally includes a battery cell 12 and a degradation determination system 14. The system 10 is configured to determine and/or predict degradation of the battery cell 12 using the degradation determination system 14. In the case of a lithium manganese-rich (LMR) battery cell 12, the LMR is susceptible to degradation related to voltage decay and other side reactions that lead to rapid cathode deterioration and loss of cell voltage. LMR degradation modes are not currently quantified and accurate battery cell models for performance (e.g., capacity prediction and battery state estimation) may be difficult to develop. Hence, the system 10 and method disclosed herein are configured to diagnose and predict different degradation mechanisms of LMR-based battery cells under normal operating conditions including OCV decay, gas generation, SEI/CEI growth, and cell resistance increase, which cause capacity loss. Additionally, the system 10 provides a high-fidelity physics-based LMR-based cell model that can be used to simulate cell performance metrics.

The battery cell 12 is used to store electrical energy in the form of chemical energy. The battery cell 12 disclosed herein is generally a lithium-ion battery cell, and more specifically a lithium ion battery cell 12 with a manganese-rich (LMR) cathode. In other examples, the battery cell 12 is a lithium-ion battery cell with other cathode materials (e.g., a lithium cobalt oxide (LiCoO₂) battery cell, a lithium manganese oxide (LiMn₂O₄) battery cell, a lithium iron phosphate (LiFePO₄) battery cell, a lithium nickel cobalt aluminum oxide (LiNiCoAlO₂ or NCA) battery cell, a lithium nickel manganese cobalt oxide (LiNiMnCoO₂ or NMC) battery cell, a lithium titanate (Li₄Ti₅O₁₂) battery cell, and the like). It should be understood that the battery cell 12 may utilize other cell chemistries besides lithium-Ion without departing from the scope of the present disclosure. In an exemplary embodiment, the battery cell 12 includes a cathode 16, an anode 18, an electrolyte 20 in contact with the cathode 16 and the anode 18, and a gas sensor 22.

The cathode 16 provides a source of lithium ions and determines capacity and average voltage of a battery. In an example, the cathode 16 is made of a mixed metal oxide of lithium, nickel, manganese, and/or cobalt. For example, the cathode 16 may be an LMR cathode including a material having the formula xLi₂MnO₃·(1-x)LiMO₂, where M represents transition metals such as nickel (Ni), cobalt (Co), manganese (Mn), and so forth, and where x is a proportion of the lithium-manganese oxide component. It will be appreciated that the cathode 16 may include other materials suitable for forming a cathode.

The anode 18 stores and releases lithium ions received from the cathode when energy is needed. In an example, the anode 18 is formed of graphite, silicon oxide (SiOx), silicon (Si), combinations thereof, or any like material. It will be appreciated that the anode 18 may include other materials suitable for forming an anode.

The electrolyte 20 provides a medium between the cathode and anode through which the lithium ions travel. In an example, the electrolyte 20 includes a lithium salt (e.g., lithium hexafluorophosphate (LiPF₆), lithium bis(trifluoromethanesulfonyl)imide (LiTFSI), lithium perchlorate (LiClO₄), and the like) dissolved in a solvent (e.g., fluoroethylene carbonate (FEC), ethylene carbonate (EC), and dimethyl carbonate (DMC)). It will be appreciated that the electrolyte 20 may include other materials suitable for forming an electrolyte.

The gas sensor 22 senses and detects gases within the battery cell 12. The gas sensor 22 may include an electrochemical gas sensor, for example, which converts a presence of gas into an electrical signal that can be processed and interpreted into an electrical signal. An electrochemical sensor can detect gas through a chemical reaction that is converted to an electrical current. Other examples of a gas sensor 22 may include a semiconductor gas sensor, an infrared gas sensor (e.g., a carbon dioxide (CO₂) sensor, a methane (CH₄) sensor), a photoionization sensor for detecting volatile compounds, and/or a catalytic bead sensor. It will be appreciated that a variety of suitable gas sensors may be used as the gas sensor 22 within the battery cell 12.

During discharge, when a load is applied to a battery cell 12, Li+ ions move from the anode 18 to the cathode 16 by way of the electrolyte 20, and electrons (e) move from the cathode 16 to the anode 18 to provide energy to the battery load. While charging and upon application of an external voltage, Li+ ions move from the cathode 16 to the anode 18 by way of the electrolyte 20 and may be intercalated into the anode 18. A positive terminal 24 (or cathode current collector) and a negative terminal 26 (or anode current collector) allow the battery cell 12 to be connected to other systems for the purpose of measuring one or more states of the battery cell 12 and/or providing power to an external device.

While a battery cell 12 with an LMR cathode is generally stable, the LMR cathode is susceptible to degradation related to voltage decay and other side reactions that lead to rapid cathode deterioration and loss of cell voltage. LMR cathode degradation modes are currently not quantified and accurate battery cell models have not been developed for performance, for example for capacity prediction and battery state estimation.

In the scope of the present disclosure, a state of charge (SOC) generally refers to an amount or concentration of lithium ions intercalated within a lithium ion intercalation material (i.e., a material capable of intercalating lithium ions) relative to a maximum capacity of the lithium ion intercalation material to intercalate lithium ions. A state of charge (SOC) of the battery cell 12 quantifies a present level of charge stored in the battery cell 12 relative to a maximum charge capacity of the battery cell 12. On a molecular level, the SOC of the battery cell 12 refers to the distribution of lithium ions between the cathode 16 and the anode 18. More particularly, the SOC of the battery cell 12 quantifies an amount or concentration of lithium ions intercalated within the anode 18 relative to a maximum capacity of the anode 18 to intercalate lithium ions. In an example, when the battery cell 12 is fully charged (i.e., the SOC of the battery cell 12 is 100%), the anode 18 is fully intercalated with lithium ions. As the battery cell 12 discharges, lithium ions move from the anode 18 to the cathode 16 through the electrolyte 20 resulting in a decrease in the concentration of lithium ions in the anode 18 and an increase in the concentration of lithium ions in the cathode 16, thus decreasing the SOC of the battery cell 12.

Over charging or over discharging the battery cell 12 may damage components of the battery cell 12, such as the cathode 16 and/or the anode 18, resulting in a reduction of an overall usable life of the battery cell 12. Therefore, it is advantageous to determine the SOC of the battery cell 12 for the purposes of battery management. Generally, the SOC of the battery cell 12 is not a directly measurable quantity, and instead must be estimated using a mathematical model of the electrochemical processes occurring within the battery cell 12. In a non-limiting example, the mathematical model is configured to determine or estimate the SOC of the battery cell 12 based at least in part on an open circuit voltage (OCV) of the battery cell 12.

Open-circuit voltage (OCV) is a voltage of the battery cell 12 when not connected to a load and when there is no current. OCV is a crucial parameter for understanding a battery's state of charge (SOC) and state of health (SOH). A higher OCV generally indicates a higher SOC. Open-circuit voltage (OCV) hysteresis is when the OCV of a battery differs depending on whether it is being charged or discharged. This effect is particularly noticeable in lithium-ion batteries.

Solid Electrolyte Interphase (SEI) is a crucial component in lithium-ion batteries. The SEI (or SEI layer) forms on a surface of the anode 18 during initial charge and discharge cycles. The decomposition of the electrolyte 20 occurs at characteristic voltages and is accompanied by production of gases, which must be vented from the battery cell 12. The gases produced by the cell formation process can also provide data that may be used to assess the quality of the battery cell 12. Excessive production of gas can be indicative of a low-quality battery cell 12. Excessive gases may be due to several reasons. As one example, the complete inactivity of electrolyte additives, such as vinyl carbonate (VC), vinyl ethylene carbonate (VEC), etc., can lead to excessive consumption of ethylene carbonate (EC) resulting in gas production. In this situation, the battery cell 12 shows very poor charge retention with cycling. Additionally, poor additive performance due to partial expiration and degradation can also lead to excessive EC consumption and increased gas generation volume.

The SEI is a thin film, typically about 100-120 nanometers (nm) thick, and is composed of various inorganic and organic compounds (e.g., lithium carbonate (Li₂CO₃), lithium fluoride (LiF), and lithium alkyl carbonates (ROCO₂Li). The SEI plays a significant role in the battery's performance and longevity and allows lithium ions to pass through while blocking electrons, which helps prevent further reactions that could degrade the battery cell 12. The SEI improves the cycling performance and extends the battery's life by protecting the electrode materials. A common reason for a low-quality battery cell is an insufficient Solid Electrolyte Interphase (SEI) deposited on the anode 18 of the battery cell 12.

Cathode-Electrolyte Interphase (CEI) is a critical layer that forms on a surface of the cathode 16 in lithium-ion batteries. Similar to the SEI on the anode 18, the CEI is essential for the battery's performance and longevity. The CEI layer is formed by reactions between the cathode 16 and the electrolyte 20 during battery operation. This layer helps stabilize the cathode by preventing further unwanted reactions, which can degrade the battery over time.

Gases produced by the battery cell 12 can provide data used to assess quality of the battery cell 12. Excessive production of gas can be indicative of a low-quality battery cell 12. Excessive gas may be due to several reasons. For example, complete inactivity of electrolyte additives, such as vinyl carbonate (VC), vinyl ethylene carbonate (VEC), and so forth, leads to excessive consumption of ethylene carbonate (EC) resulting in gas production. In this situation, a battery cell 12 shows poor charge retention with cycling. Poor electrolyte additive performance due to partial expiration and degradation also leads to excessive EC consumption and increased gas generation volume.

In general, a small gas volume in the battery cell 12 results in the highest charge capacity of the battery cell 12, while an increase in gas volume (e.g., due to EC reduction) is correlated to degradation of charge capacity over time. Excessive ethylene carbonate (EC) reduction consumes lithium salt in the electrolyte, which lowers the total available “lithium inventory” in the battery cell 12, which reduces ultimate charge capacity. Moreover, poor electrolyte additive performance causes a more rapid breakdown of the SEI layer. As a result, additional EC reduction is necessary to maintain the SEI layer. The SEI layer formed primarily from EC reduction has poor mechanical properties and greater thickness, which is inferior to one formed when electrolyte additives are present.

Referring again to FIG. 1, the degradation determination system 14 is used to determine and quantify degradation of an LMR cathode and battery cell 12. The degradation determination system 14 is in electrical communication with the positive terminal 24 (cathode current collector) and the negative terminal 26 (anode current collector). In one embodiment, the degradation determination system 14 is physically coupled and affixed to the battery cell 12 (or battery pack) so that the degradation determination system 14 may operate even when the battery cell 12 is not installed in the battery pack. While the degradation determination system 14 is shown in FIG. 1 as being affixed to the battery cell 12, it will be understood that the degradation determination system 14 may be integrated into a housing of the battery cell 12 (or battery pack), disposed within the battery cell 12 with internal connections to the cathode 16 and the anode 18, or otherwise integral with the battery cell 12 without departing from the spirit and scope of the present disclosure.

In another example, the degradation determination system 14 may be a modular component configured to be removable, installable, and replaceable on the battery cell 12. In another example, the degradation determination system 14 is located remotely from the battery cell 12 and in electrical communication with the battery cell 12.

Referring to FIG. 2, a schematic diagram of the degradation determination system 14 is shown. In an example, the degradation determination system 14 includes at least a controller 28 and an interface circuit 30.

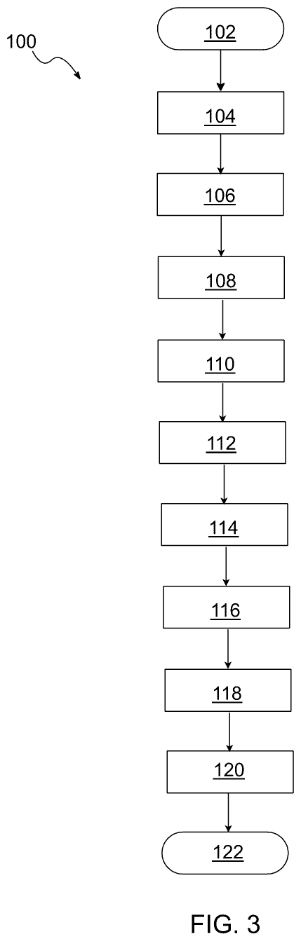

The controller 28 is used to implement a method 100, as illustrated in FIG. 3, for determining lithium manganese-rich (LMR) battery cell quality, as will be described below. The controller 28 includes at least one processor 32 and a non-transitory computer readable storage device or memory 34. The processor 32 may be a custom made or commercially available processor, a central processing unit (CPU), a graphics processing unit (GPU), an auxiliary processor among several processors associated with the controller 28, a semiconductor-based microprocessor (in the form of a microchip or chip set), a macroprocessor, a field programmable gate array (FPGA), an application specific integrated circuit (ASIC), a combination thereof, or generally a device for executing instructions.

The computer readable storage device or memory 34 may include volatile and nonvolatile storage in read-only memory (ROM), random-access memory (RAM), and keep-alive memory (KAM), for example. KAM is a persistent or non-volatile memory that may be used to store various operating variables while the processor 32 is powered down. The computer-readable storage device or memory 34 may be implemented using a number of memory devices such as PROMs (programmable read-only memory), EPROMs (electrically PROM), EEPROMs (electrically erasable PROM), flash memory, or another electric, magnetic, optical, or combination memory devices capable of storing data, some of which represent executable instructions used by the controller 28 to control the degradation determination system 14. The controller 28 may also consist of multiple controllers which are in electrical communication with each other. The controller 28 further may include additional elements and/or modules, such as, for example, a real-time clock (RTC) module for measuring the passage of real-time. In an exemplary embodiment, the controller 28 is powered by connection to the battery cell 12.

The controller 28 is in electrical communication with the interface circuit 30. In an exemplary embodiment, the electrical communication is established using, for example, general purpose input/output (GPIO) pins, an inter-integrated circuit (I2C) bus, a serial peripheral interface (SPI) bus, a parallel communication bus, or the like. It should be understood that various additional communication protocols for communicating with the controller 28 are within the scope of the present disclosure.

The interface circuit 30 is used to interface the controller 28 with the positive terminal 24 (cathode current collector) and the negative terminal 26 (anode current collector). In an exemplary embodiment, the interface circuit 30 includes an OCV measurement circuit 36 and a gas sensing circuit 38.

The OCV measurement circuit 36 is used to measure voltage and OCV shifting of the LMR battery cell 12 during cycling based on a nonlinear voltage decay rate of LMR material. In a non-limiting example, the OCV measurement circuit 36 includes, for example, an analog to digital converter (ADC). The OCV measurement circuit 36 may further include additional components to support voltage measurement, including, for example, a voltage follower, an input buffer, a multiplexer, and/or the like. The OCV measurement circuit 36 further includes components allowing the controller 28 to measure the current flow into/out of the positive terminal 24 (cathode current collector) and/or the negative terminal 26 (anode current collector), including, for example, a shunt resistor, an electromagnetic current sensor, an ADC, and/or the like.

The gas sensing circuit 38 is used to receive, interpret, process, and/or measure electrical signals from the gas sensor 22. The gas sensing circuit 38 is in electrical communication with the gas sensor 22 within the battery cell 12. In an exemplary embodiment, the gas sensing circuit 38 includes a signal conditioning circuit (e.g., an operational amplifier, a low-pass filter, and/or an analog-to-digital converter (ADC) and/or a microcontroller or microprocessor. In some examples, the gas sensing circuit 38 includes, for example, relays, transistors, and/or the like. It should be understood that the OCV measurement circuit 36 and/or the gas sensing circuit 38 of the interface circuit 30 may further include additional passive or active analog and/or digital electronics such as, for example, resistors, capacitors, inductors, filters, amplifiers, power electronics, digital to analog converters (DAC), and/or the like. In an example, the interface circuit 30 is powered by connection to the positive terminal 24 (cathode current collector) and/or the negative terminal 26 (anode current collector) of the battery cell 12. Additionally, the interface circuit 30 is in electrical communication with the controller 28.

Referring to FIG. 3, a flowchart of a method 100 for determining lithium manganese-rich (LMR) battery cell quality is shown. The method begins at block 102.

Block 102 depicts measuring open circuit voltage (OCV) of a battery cell during cycling to obtain testing data. Controller 28 can cause the interface circuit 30 and the OCV measurement circuit 36 to collect raw LMR testing data and measure voltage from the positive terminal 24 (cathode current collector) and the negative terminal 26 (anode current collector) for determining OCV shifting of the LMR battery cell 12 during cycling based on a nonlinear decay rate of the LMR material (e.g., material of the cathode 16 and/or the anode 18). For example, the OCV measurement circuit 36 may measure or obtain an OCV scan every 10 charge and discharge cycles during a first 50 cycles of the battery life and every 100 cycles until an end of life of the battery. Defining a life cycling operation may include charging the battery cell by increasing a cell voltage up to approximately 4.2 volts and discharging the battery cell to reduce the cell voltage from approximately 4.2 volts down to approximately 2.7 volts. In this context, one of skill in the art would understand the term “approximately.” Alternatively, the term “approximately” is understood to mean plus or minus 0.1 volts. In an example, three-electrode testing may be used to cycle LMR/graphite coin and pouch cells using a C/5 current with a C/100 OCV measurement between every 10-100 cycles to identify LMR and full-cell OCV shift. In another example, the OCV measurement circuit 36 can measure and scan cycled LMR half-coin cells with different lower and upper cut-off voltages to identify LMR OCV hysteresis changes to determine baseline LMR OCV measurements. Further, the OCV measurement circuit 36 may perform mini-sweep cycling at different C-rates to characterize voltage hysteresis transit.

Block 104 depicts predicting OCV shifting by integrating the testing data into a multi-site multi-reaction (MSMR) framework to obtain MSMR data. Predicting may include using the controller 28 and/or the interface circuit 30 to implement an analytical tool that smooths test data obtained at block 102 and generates dQ/dV curves for peak location identification. The peak location shifts in the dQ/dV curves can be used to quantify OCV shifts and to determine OCV hysteresis gaps and midpoints. For example, the dQ/dV curves and peak location identification can be determined using the following two equations, where the fractional Li occupancy x is described as a function of voltage U, and where the differential dx/dU can be used to determine peaks which indicate individual reactions/galleries. In these equations, f is a scaling factor and w; is a width parameter for each component.

$$x ( U ) = {\sum }_{j}^{{N}_{r}} {x}_{j} ( U ) = {\sum }_{j}^{{N}_{r}} \frac{{X}_{j}}{1 + exp [ \frac{f ( U - {U}_{j}^{0}}{{ω}_{j}} ]}$$

$$\frac{dx}{dU} = {\sum }_{j}^{{N}_{r}} \frac{{dx}_{j}}{dU} = {\sum }_{j}^{{N}_{r}} - \frac{{X}_{j}}{{ω}_{j}} \frac{f exp [ f ( U {-}_{j}^{0} / {ω}_{j} ]}{{ω}_{j} {\{ 1 + exp [ U - {U}_{j}^{0} ] / {ω}_{j} \}}^{2}}$$

An OCV hysteresis midpoint and gap can be defined using the following equation.

$${U}_{mid} ( x ) = \frac{1}{2} ( {U}_{discharge} ( x ) + {U}_{charge} ( x ) )$$

$${U}_{gap} ( x ) = \frac{1}{2} ( {U}_{discharge} ( x ) + {U}_{charge} ( x ) )$$

Fitting the following equation to mini-sweep data can be used to calculate OCV transit parameters, where −1≤ζ≤1 is the OCV transit variable and K is a constant fitted to mini-sweep test data.

$$U ( x ) = {U}_{mid} ( x ) + {U}_{gap} ( x ) ζ$$

$$\frac{d ζ}{dx} = - K [ 1 + sign ( \frac{dx}{dt} ) ζ ]$$

Block 106 depicts determining OCV hysteresis changes of the LMR battery cell at different phases of voltage activation of the battery cell. For example, OCV measurement circuit 36 and/or controller 28 can quantify OCV hysteresis changes of LMR at different phases (e.g., layered→spinel) by measuring voltage activation at specific cut-off voltages (e.g., at 3.8V, 4.0V, 4.2V, 4.4V, and/or 4.6V).

Block 108 depicts determining cell voltage decay with an accurate state of charge (SOC) using the MSMR data obtained at block 104 and the OCV hysteresis changes obtained at block 106. The controller 28 can determine the cell voltage decay using accurate state of charge (SOC) measurements over a cycle life of the battery cell 12 from the OCV model used in block 104 with the rate-invariant hysteresis across many cycles determined in block 106 to obtain degradation metrics that reflect structure changes within the battery cell 12.

Block 110 depicts measuring carbon dioxide (CO₂) within the battery cell 12 to determine a set of electrolyte consumption data for the battery cell 12. As the electrolyte 20 in the battery cell decomposes over time, CO₂ is produced. A measured amount of CO₂ can be correlated with electrolyte self-decomposition and decrease in electrolyte volume to identify dry out conditions and loss of utilized electrode area. In an example, controller 28 and/or interface circuit 30 can cause the gas sensor 22 to detect an amount of CO₂ within the battery cell 12 every 100 charging cycles. Additionally, controller 28 can correlate the CO₂ with degradation of the electrolyte 20.

Block 112 depicts measuring gas compositions within the battery cell 12 to determine a set of solid electrolyte interphase (SEI)/cathode electrolyte interphase (CEI) growth and lithium (Li) plating data for the battery cell 12. Certain gas compositions existing within the battery cell 12 indicate and can be correlated with SEI growth, lithium (Li) plating, and electrolyte consumption. Some examples of gases measured by the gas sensor 22 can include at least one of ethylene (C₂H₄), ethane (C₂H₆), hydrogen gas (H₂), or a combination thereof. In an example, the controller 28 and/or the gas sensing circuit 38 can cause the gas sensor 22 to measure and/or detect the gas compositions every 100 cycles. In some instances, identification of the gas compositions may be identified using, for example, nuclear Magnetic Resonance (NMR) spectroscopy analysis.

Block 114 depicts estimating a loss of cyclable active material (LAM) by using stoichiometric ratio shifts between the cathode 16 and the anode 18 in the battery cell 12 based on the testing data to obtain a set of reaction rate constants. For example, the controller 28 can use stoichiometric ratio shifts between the anode 18 and the cathode 16 to identify utilized electrode capacity ranges and loss of cyclable active material (LAM) on the electrodes using the test data obtained from the cycling measurements. LAM is a degradation process in the battery cell 12 where active material that participate in electrochemical reactions become inactive or unavailable for cycling. LAM may occur due to electrode cracking, side reactions, and/or phase transitions. These factors can contribute to overall capacity fade of the battery cell 12.

Block 116 depicts fitting the set of reaction rate constants to a rate limited kinetic model and a diffusion limited model to obtain a set of kinetic model parameters and to obtain a set of diffusion model parameters. For example, controller 28 can use the following equations to determine rate limited kinetic models for SEI growth and lithium (Li) plating, where j_{SEI} is a current density related to SEI formation, k_{SEI} is rate constant for the SEI formation reaction, c_{EC} is a concentration of the electrolyte component (e.g., ethylene carbonate (EC)), a is a charge transfer coefficient, F is Faraday's constant, R is a universal gas constant, T is a temperature in Kelvin, φ_{S} is a potential of the solid electrode, de is a potential of the electrolyte, jₜₒₜ is total current density, R_{film} is resistance of the SEI film, and User is equilibrium potential of the SEI.

$${j}_{SEI} = - {k}_{SEI} {c}_{EC} exp [ - \frac{α F}{RT} ( {ϕ}_{s} - {ϕ}_{e} - {j}_{tot} {R}_{film} - {U}_{SEI} ) ]$$

Controller 28 can use the following equation (diffusion limited model) to determine electrolyte solvent decomposition, where j_{SEI,diff} is diffusion-limited current density related to SEI formation, F is Faraday's constant, aₙ is a specific surface area of the electrode,

$${D}_{EC}^{SEI}$$

is a diffusion coefficient of the electrolyte component (e.g., ethylene carbonate (EC)) within the SEI, c_{EC} is concentration of the electrolyte component, and δ_{SEI} is thickness of the SEI layer.

$${j}_{SEI , diff} = \frac{{Fa}_{n} {D}_{EC}^{SEI} {c}_{EC}}{{δ}_{SEI}}$$

Fitting the set of reaction rate constants to a rate limited kinetic model and a diffusion limited model to obtain a set of kinetic model parameters and to obtain a set of diffusion model parameters can include minimizing squared errors between cell capacities from simulation and test measurements.

Block 118 depicts determining solid electrolyte interphase (SEI) and metallic Li thicknesses on the anode and cathode electrolyte interphase (CEI) thickness on the cathode 16 using the kinetic model parameters obtained at block 116. A decrease in electrolyte (e.g., EC) volume and pore volume due to EC consumption can be determined by controller 28 using the following equations, where

$${dV}_{e}^{JR} {dV}_{EC}$$

are differential volumes of the electrolyte 20, dn_{EC} is a differential amount of the electrolyte 20, M_{EC} IS molar mass of the electrolyte 20, and ρ_{EC} is density of the electrolyte 20.

$${dV}_{e}^{JR} = {dV}_{EC} = {dn}_{EC} \frac{{M}_{EC}}{{ρ}_{EC}}$$

Controller 28 can determine a thickness change of the SEI layer using the following equation, where

$$\frac{d {δ}_{SEI}}{dt}$$

is rate of change of the SEI layer thickness with respect to time, j_{SEI} is current density related to SEI formation, aₙ is specific surface area of the cathode, F is Faraday's constant, M_{SEI} is molar mass of the SEI layer, and ρ_{SEI} is density of the SEI material.

$$\frac{d {δ}_{SEI}}{dt} = - \frac{{j}_{SEI}}{2 {a}_{n} F} \cdot \frac{{M}_{SEI}}{{ρ}_{SEI}}$$

Block 120 depicts determining cell resistance and voltage drop using the SEI and metallic Li thicknesses and porosity decrease. Controller 28 can determine a decrease in electrode porosity due to thickening of the SEI layer using the following equation, where

$$\frac{d {ε}_{n}}{dt}$$

is rate of change of porosity over time, aₙ is specific surface area of the cathode, and

$$\frac{d {δ}_{SEI}}{dt}$$

is rate of change of the SEI layer thickness over time.

$$\frac{d {ε}_{n}}{dt} = - {a}_{n} \frac{d {δ}_{SEI}}{dt}$$

Controller 28 can determine an increase in resistance due to SEI growth using the following equation, where R_{SEI} is resistance of the SEI layer, εₙ is porosity of the SEI layer, δ_{SEI} is thickness of the SEI layer, and κ_{SEI} is ionic conductivity of the SEI layer.

$${R}_{SEI} = \frac{{ε}_{n} {δ}_{SEI}}{{κ}_{SEI}}$$

Block 122 depicts predicting performance metrics using an electrochemical model. The performance metrics may include a remaining useful battery cell life, a battery cell state of health (SOH), a cell voltage evolution, and a cell resistance and impedance. In an example, the electrochemical model may include a pseudo-two-dimensional (P2D) model, also known as the Newman model. The P2D model simplifies a three dimensional structure of a battery into a two-dimensional framework making determination of performance metrics computationally efficient while still capturing essential electrochemical processes. The P2D model can use the determined and obtained OCV with hysteresis, the reaction models for SEI and CEI growth and Li plating, the stoichiometry shifts for loss of cyclable active material (LAM), and the electrolyte volume decrease to predict the remaining useful cell life, the battery state of health (SOH), cell voltage evolution, and cell resistance and impedance.

The system 10 and method 100 for predicting and determining LMR battery cell degradation of the present disclosure offers several advantages. The LMR battery cell is susceptible to degradation related to voltage decay and other side reactions that lead to rapid cathode deterioration and loss of cell voltage. The system 10 and method 100 are configured to diagnose and predict different degradation mechanisms of LMR-based battery cells under normal operating conditions including OCV decay, gas generation, SEI/CEI growth, and cell resistance increase, which cause capacity loss. Additionally, the system 10 provides a high-fidelity physics-based LMR-based cell model that can be used to simulate cell performance metrics.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. A method for determining degradation of a battery cell with a lithium-manganese-rich (LMR) cathode, comprising:
measuring open circuit voltage (OCV) of a battery cell during cycling to obtain testing data;
predicting OCV shifting by integrating the testing data into a multi-site multi-reaction (MSMR) framework to obtain MSMR data;
determining OCV hysteresis changes of the battery cell at different phases of voltage activation of the battery cell;
determining cell voltage decay with an accurate state of charge (SOC) using the MSMR data and the OCV hysteresis changes;
measuring carbon dioxide (CO₂) within the battery cell to determine a set of electrolyte consumption data for the battery cell;
measuring gas compositions within the battery cell to determine a set of solid electrolyte interphase (SEI)/cathode electrolyte interphase (CEI) growth and lithium (Li) plating data for the battery cell;
estimating a loss of cyclable active material (LAM) by using stoichiometric ratio shifts between a cathode and an anode in the battery cell based on the testing data to obtain a set of reaction rate constants;
fitting the set of reaction rate constants to a rate limited kinetic model and a diffusion limited model to obtain a set of kinetic model parameters and to obtain a set of diffusion model parameters;
determining solid electrolyte interphase (SEI) and metallic Li thicknesses on the anode and cathode electrolyte interphase (CEI) thickness on the cathode using the kinetic model parameters;
determining cell resistance and voltage drop using the solid electrolyte interphase (SEI) and metallic Li thicknesses and porosity decrease; and
predicting performance metrics using an electrochemical model to obtain a remaining useful battery cell life, battery cell state of health (SOH), cell voltage evolution, and cell resistance and impedance.

2. The method of claim 1, wherein measuring the open circuit voltage (OCV) of a battery cell during cycling includes performing an OCV scan once every 10 cycles for a first 50 cycles and then performing an OCV scan once every 100 cycles until an end of life of the battery cell to obtain testing data.

3. The method of claim 1, wherein measuring the open circuit voltage (OCV) of a battery cell during cycling includes three-electrode testing, LMR half-cell OCVs, and mini-sweep cycling.

4. The method of claim 1, wherein predicting OCV shifting by integrating the testing data into a multi-site multi-reaction (MSMR) framework to obtain MSMR data includes smoothing test data and generating dQ/dV curves for peak identification.

5. The method of claim 4, wherein predicting OCV shifting includes quantifying OCV shifts based on peak location shifts in dQ/dV curves and determining OCV hysteresis gaps and midpoints.

6. The method of claim 1, wherein determining OCV hysteresis changes of the LMR battery cell at different phases of voltage activation of the battery cell includes determining OCV hysteresis changes at 3.8 volts, 4.0 volts, 4.2 volts, 4.4 volts, and at 4.6 volts.

7. The method of claim 1, wherein estimating a loss of cyclable active material (LAM) includes correlating CO₂ volume with electrolyte self-decomposition and a decrease in electrolyte volume to identify dry out conditions and loss of utilized electrode area.

8. The method of claim 1, wherein estimating a loss of cyclable active material (LAM) includes correlating measured gas compositions with SEI growth, Li plating, and consumed electrolyte using NMR analysis, and wherein the gas compositions include at least one of ethylene (C₂H₄), ethane (C₂H₆), or hydrogen gas (H₂).

9. The method of claim 1, wherein fitting the set of reaction rate constants to a rate limited kinetic model and a diffusion limited model to obtain a set of kinetic model parameters and to obtain a set of diffusion model parameters includes minimizing squared errors between cell capacities from simulation and test measurements.

10. The method of claim 1, further including:
defining a life cycling operation to include:
charging the battery cell by increasing a cell voltage up to approximately 4.2 volts; and
discharging the battery cell to reduce the cell voltage from approximately 4.2 volts down to approximately 2.7 volts.

11. The method of claim 1, wherein the cathode is formed from a material having the formula xLi₂MnO₃·(1-x)LiMO₂, where M represents at least one of nickel (Ni), cobalt (Co), or manganese (Mn), and where x is a proportion of a lithium-manganese oxide component.

12. The method of claim 1, wherein the anode is formed from at least one of graphite, SiOx, or Si.

13. A method for determining quality of a battery cell having a lithium manganese-rich (LMR) cathode, comprising:
measuring open circuit voltage (OCV) of a battery cell to obtain testing data;
integrating the testing data into a multi-site multi-reaction (MSMR) framework to obtain MSMR data and to predict OCV shifting;
determining OCV hysteresis changes of the LMR battery cell at different phases of voltage activation of the battery cell;
determining cell voltage decay with an accurate state of charge (SOC) using the MSMR data and the OCV hysteresis changes;
measuring gas compositions every 100 charging cycles of the battery cell to obtain a set of reaction rate constants;
determining a set of electrolyte consumption data and a set of SEI/CEI growth and lithium (Li) plating data for the battery cell based on the set of reaction rate constants;
determining a loss of cyclable active material (LAM) by obtaining a set of stoichiometric coefficients using stoichiometric ratio shifts between a cathode and an anode in the battery cell; and
determining cell resistance and voltage drop, remaining useful battery cell life, and battery cell state of health (SOH) based on the SEI/CEI growth and lithium (Li) plating data and based on the MSMR data and the OCV hysteresis changes.

14. The method of claim 13, wherein measuring the open circuit voltage (OCV) of a battery cell includes performing an OCV scan once every 10 cycles for a first 50 cycles and then performing an OCV scan once every 100 cycles until an end of life of the battery cell to obtain the testing data.

15. The method of claim 13, wherein determining OCV hysteresis changes includes smoothing test data and generating dQ/dV curves for peak identification.

16. The method of claim 13, wherein determining OCV hysteresis changes of the LMR battery cell includes determining OCV hysteresis changes at 3.8 volts, 4.0 volts, 4.2 volts, 4.4 volts, and at 4.6 volts.

17. The method of claim 13, wherein determining a loss of cyclable active material (LAM) includes correlating CO₂ volume with electrolyte self-decomposition and a decrease in electrolyte volume to identify dry out conditions and loss of utilized electrode area.

18. The method of claim 13, wherein the cathode is formed from a material having the formula xLi₂MnO₃·(1-x)LiMO₂, where M represents at least one of nickel (Ni), cobalt (Co), or manganese (Mn), and where x is a proportion of a lithium-manganese oxide component.

19. The method of claim 13, wherein the anode is formed from at least one of graphite, silicon oxide (SiOx), or silicon (Si).

20. A method for determining quality of a battery cell having a lithium manganese-rich (LMR) cathode, comprising, comprising:
integrating open circuit voltage (OCV) measurements of a battery cell into a multi-site multi-reaction (MSMR) framework to obtain MSMR data and to predict OCV shifting;
determining cell voltage decay with an accurate state of charge (SOC) using the MSMR data and OCV shifting;
measuring gas compositions during charging cycles of the battery cell to obtain a set of reaction rate constants;
determining a set of electrolyte consumption data and a set of SEI/CEI growth and lithium (Li) plating data for the battery cell based on the set of reaction rate constants;
determining a loss of cyclable active material (LAM) by finding a set of stoichiometric coefficients using stoichiometric ratio shifts between a cathode and an anode in the battery cell; and
determining cell resistance and voltage drop, remaining useful battery cell life, and battery cell state of health (SOH) based on the SEI/CEI growth and lithium (Li) plating data and based on the MSMR data and OCV hysteresis changes.
